(19) United States
(12) Patent Application Publication
(10) Pub. No.: US 2014/0243850 A1
(43) Pub. Date: Aug. 28, 2014

(54) SURGICAL INSTRUMENT WITH CURVED JAWS FOR SURGICAL SYSTEM

(71) Applicant: INTUITIVE SURGICAL OPERATIONS, INC., Sunnyvale, CA (US)

(72) Inventor: Alain SADAKA, Sunnyvale, CA (US)

(73) Assignee: INTUITIVE SURGICAL OPERATIONS, INC., Sunnyvale, CA (US)

(21) Appl. No.: 14/192,078

(22) Filed: Feb. 27, 2014

Related U.S. Application Data
(60) Provisional application No. 61/770,785, filed on Feb. 28, 2013.

(51) Int. Cl. A61B 19/00

(52) U.S. Cl. CPC A61B 19/2203
USPC 606/130

(57) ABSTRACT

A teleoperated surgical system including a rigid cannula and a surgical instrument is provided. The rigid cannula may include at least a portion having a curved longitudinal axis. The teleoperated surgical system may include a force transmission mechanism configured to engage a patient side manipulator of the teleoperated surgical system, a flexible shaft, and an end effector coupled to the shaft. The end effector may have a shape enhancing visibility of the end effector and/or enhancing visibility of procedures performed with the end effector. Further, the shape of the end effector may enhance grasping of objects. The end effector may comprise a pair of jaws. At least a portion of each jaw may have a curved longitudinal axis. A surgical instrument and a teleoperated surgical system including a surgical instrument and a needle are also provided.

This application claims the benefit of U.S. Provisional Application No. 61/770,785, filed Feb. 28, 2013, which is incorporated by reference herein in its entirety.

### TECHNICAL FIELD

Aspects of the present disclosure relate to surgical instruments that comprise jawed end effectors. In particular, aspects of the present disclosure relate to such surgical instruments for use in teleoperated surgical systems.

### INTRODUCTION

Some minimally invasive surgical techniques are performed remotely through the use of teleoperated (robotically-controlled) surgical instruments. In teleoperated (robotic) surgical systems, surgeons manipulate input devices at a surgeon console, and those inputs are passed to a patient side cart that interfaces with one or more teleoperated surgical instruments. Based on the surgeon's inputs at the surgeon console, the one or more teleoperated surgical instruments are actuated at the patient side cart to operate on the patient, thereby creating a master-slave control relationship between the surgeon console and the surgical instrument(s) at the patient side cart.

The patient side cart of a teleoperated surgical system may have multiple arms to which a plurality of teleoperated surgical instruments may be coupled. When the surgical instruments are used to operate on a patient, it may be desirable to minimize the number and/or extent of surgical incisions in a patient to reduce the invasiveness of a surgical procedure. To accomplish this, it may be desirable to minimize the size of surgical instruments and their associated movements. When addressing these considerations, the arrangement of the surgical instruments may be considered, particularly how a surgeon is able to view the surgical instruments when they reach the surgical site within the patient's body at which a procedure is to be performed.

### SUMMARY

Exemplary embodiments of the present disclosure may solve one or more of the above-mentioned problems and/or may demonstrate one or more of the above-mentioned desirable features. Other features and/or advantages may become apparent from the description that follows.

In accordance with at least one exemplary embodiment, a teleoperated surgical system comprises a rigid cannula and a surgical instrument. The rigid cannula may include at least a portion having a curved longitudinal axis. The surgical instrument may comprise a force transmission mechanism, a flexible shaft, and an end effector. The force transmission mechanism may be configured to engage a patient side manipulator of the surgical system. The end effector may be coupled to the shaft and may comprise a pair of jaws. At least a portion of each jaw may have a curved longitudinal axis.

In accordance with at least one exemplary embodiment, a surgical instrument may comprise a force transmission mechanism, a flexible shaft, and an end effector. The force transmission mechanism may be configured to engage a patient side manipulator of a teleoperated surgical system. The end effector may be coupled to the shaft and may comprise a pair of jaws. At least a portion of each jaw may have a curved longitudinal axis.

In accordance with at least one exemplary embodiment, a teleoperated surgical system may comprise a surgical instrument and a needle. The surgical instrument may comprise a force transmission mechanism, a flexible shaft, and an end effector. The force transmission mechanism may be configured to engage a patient side manipulator of the teleoperated surgical system. The end effector may be coupled to the flexible shaft and may include a pair of jaws. At least a portion of each jaw may have a curved longitudinal axis. The needle may be configured to be grasped by the jaws.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure, and are incorporated in and constitute a part of this specification. The drawings illustrate one or more exemplary embodiments of the present teachings and together with the description serve to explain certain principles and operation.

FIG. 1 is a schematic side view of portions of patient side manipulators mounted with cannulas and surgical instruments of a teleoperated surgical system, according to an exemplary embodiment.

FIG. 2 is a schematic diagram illustrating an endoscopic camera view of two surgical instruments having straight end effectors, according to an exemplary embodiment.

FIG. 3 is a partial perspective view of an exemplary embodiment of a surgical instrument having a curved end effector.

FIG. 4 is an exploded view of the surgical instrument of FIG. 3.

FIG. 5 is a top view of the end effector of FIG. 3, along line 5-5 of FIG. 3.

FIG. 6 is a side view of the end effector of FIG. 3, along line 6-6 of FIG. 3, the end effector being in a closed position.

FIG. 7 is a view of the end effector of FIG. 6 in an open position.

FIG. 8 is a schematic diagram illustrating an endoscopic camera view of two surgical instruments having curved end effectors, according to an exemplary embodiment.

FIG. 9 is a partial side view of an exemplary embodiment of a surgical instrument inserted within a curved cannula.

FIG. 10 is a partial top view of an exemplary embodiment of a surgical instrument with a curved end effector.

FIG. 11 is a view along line 11-11 in FIG. 10.

FIG. 12 is a partial top view of an exemplary embodiment of a surgical instrument with a curved end effector.

FIG. 13 is a partial perspective view of an exemplary embodiment of a surgical instrument grasping a needle.

FIG. 14 is a side view of the end effector, needle, and suture of FIG. 13 along line 14-14 of FIG. 13.

FIG. 15 is a partial perspective view of an exemplary embodiment of a surgical instrument grasping a needle.

FIG. 16 is a side view of the end effector, needle, and suture of FIG. 15 along line 16-16 of FIG. 15.

FIG. 17 is a partial perspective view of an exemplary embodiment of a surgical instrument grasping a needle.

FIG. 18 is a partial perspective view of an exemplary embodiment of a surgical instrument grasping a needle.

FIG. 19 schematically depicts an exemplary action of a suturing procedure, according to an exemplary embodiment.

FIG. 20 schematically depicts an exemplary action of a suturing procedure, according to an exemplary embodiment.

FIG. 21 schematically depicts an exemplary action in a suturing procedure, according to an exemplary embodiment.

FIG. 22 schematically depicts an exemplary action in a suturing procedure, according to an exemplary embodiment.

FIG. 23 schematically depicts an exemplary action in a suturing procedure, according to an exemplary embodiment.

FIG. 24 schematically depicts an exemplary action in a suturing procedure, according to an exemplary embodiment.

FIG. 25 is a schematic diagram illustrating an endoscopic camera view of a surgical instrument during a knot-tying procedure, according to an exemplary embodiment.

FIG. 26 is a view along line 26-26 of FIG. 25.

FIG. 27 is a schematic diagram illustrating an endoscopic camera view of a surgical instrument during a knot-tying procedure, according to an exemplary embodiment.

FIG. 28 is a partial perspective view of a surgical instrument according to an exemplary embodiment having a wrist and a curved end effector.

### DETAILED DESCRIPTION

Exemplary embodiments discussed herein relate to surgical instruments including an end effector having a shape that may enhance visibility of the end effector and/or enhance visibility of procedures being performed with the end effector during a surgical procedure. In addition, the shape of the end effector may enhance the grasping of objects, such as for example, suturing needles, as well as advantageously minimize the size and number of incisions during a surgical procedure, such as a procedure using curved cannulas. Exemplary embodiments described herein may be used in surgical procedures, such as suturing procedures. In various exemplary embodiments, the surgical instruments described herein are configured to be used and controlled in teleoperated surgical systems.

In accordance with various exemplary embodiments, the present disclosure contemplates surgical instruments with curved end effectors that are capable of being advanced through cannulas having at least a portion defining a curved longitudinal axis.

This description and the accompanying drawings that illustrate exemplary embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Although the exemplary embodiments described herein are discussed in the context of teleoperated surgical systems, the embodiments are not limited to use with teleoperated surgical systems. For instance, the exemplary embodiments described herein may be used with other systems and surgical instruments, such as manual surgical instruments (e.g., laparoscopic), which may utilize the principles and/or end effectors described herein.

Teleoperated surgery generally involves the use of a manipulator that has multiple manipulator arms. One or more of the manipulator arms often support one or more teleoperated surgical instruments. One or more of the manipulator arms may be used to support a surgical image capture device, such as an endoscope (which may be any of a variety of structures such as a laparoscope, an arthroscope, a hysteroscope, or the like), or, optionally, some other imaging modality (such as ultrasound, fluoroscopy, magnetic resonance imaging, or the like). In various exemplary embodiments, the manipulator arms will support at least two surgical tools corresponding to the two hands of a surgeon and one image capture device. Exemplary teleoperated surgical systems are described in U.S. application Ser. No. 12/618,583, entitled “Curved Cannula Surgical System,” filed on Nov. 13, 2009 and published as U.S. Pub. No. US 2011/0071542 on Mar. 24, 2011, which is hereby incorporated by reference in its entirety.

One way to further reduce patient trauma while retaining the benefits of teleoperated surgical systems is to insert the one or more manipulator arms of the teleoperated surgical system through a single opening in a patient's body instead of inserting the arms through separate, discrete openings. The single opening may include, for example, an incision or a port feature. Further, a surgical image capture device may be inserted through the same opening as the manipulator arms. By arranging a teleoperated surgical system in this manner, fewer openings are required in a patient's body.

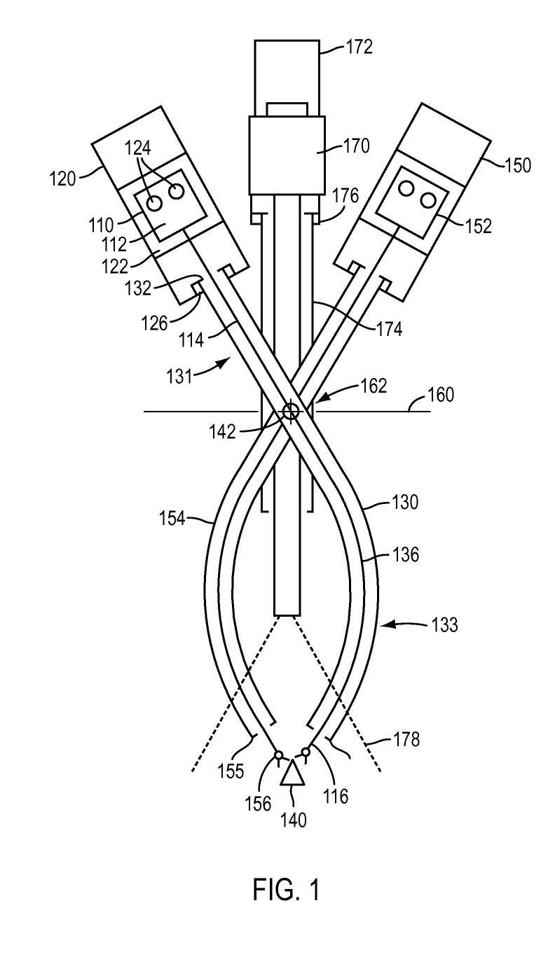

Referring now to FIG. 1, a schematic side view of a portion of robotic manipulators (that may also be referred to as a patient side manipulator or “PSM”) that support and move surgical instruments. As shown in the exemplary embodiment of FIG. 1, a teleoperated operated surgical instrument 110 includes a force transmission mechanism 112, a passively flexible shaft 114, and an end effector 116. Instrument 110 is mounted on an instrument carriage assembly 122 of a patient side manipulator 120. According to an exemplary embodiment, interface discs 124 couple actuation forces from servo actuators in patient side manipulator 120 to move instrument components.

A wrist to provide one or more end effector degrees of freedom (DOF's) (e.g., pitch, yaw; see e.g., U.S. Pat. No. 6,817,974 (filed Jun. 28, 2002) (disclosing “Surgical Tool Having Positively Positionable Tendon-Actuated Multi-Disk Wrist Joint”), which is incorporated herein by reference in its entirety) is optional and is not shown. For instance, instrument 110 may include a wrist (not shown) with a single DOF (e.g., providing pitch or yaw movement of end effector 116 relative to shaft 102). According to an exemplary embodiment, surgical instrument 110 may be a non-wristed instrument. According to an exemplary embodiment, a non-wristed instrument does not have a jointed wrist structure with at least one DOF. Omitting a wrist may reduce the number of actuation force interfaces between patient side manipulator 120 and instrument 110 and the omission also reduces the number of force transmission elements (and hence, instrument complexity and dimensions) that would be necessary between the proximal force transmission mechanism 120 and end effector 116.

FIG. 1 further shows a curved cannula 130, which has a proximal end 132, a distal end 134, and a central channel 136 that extends between proximal end 132 and distal end 134. Cannula 130 may be curved, for instance, because at least a portion of cannula 130 has a curved longitudinal axis. Cannula 130 may be curved along an entire length of cannula 130 from proximal end 132 to distal end 134 or cannula 130 may be curved along only a portion of its length, as shown in the exemplary embodiment of FIG. 1. As depicted in FIG. 1, proximal end 132 of curved cannula 130 is mounted on a cannula mount 126 of patient side manipulator 120. According to an exemplary embodiment, the constraints of patient side manipulator 120 (whether mechanical or preprogrammed software constraints in a control system for patient side manipulator 120) cause instrument 110 and curved cannula 130 to move in pitch and yaw around a remote center of motion 142 located along cannula 130. The remote center of motion 142 may be located, for example, at an incision 162 in a body wall 160 of a patient. Further, the input/output (I/O) actuation of patient side manipulator 120, provided by carriage 122, may insert and withdraw instrument 110 through cannula 130 to move end effector 116 in and out of the cannula 130 at the distal end 134. The shaft 114 of instrument 110 may be flexible to permit instrument 110 to be inserted and withdrawn through curved cannula 130 but also be sufficiently stiff to provide effective surgical action at a surgical site 140. As a result, flexible shaft 114 of instrument 110 may be extended through the central channel 136 of curved cannula 130 so that a distal portion of flexible shaft 114 and end effector 116 extend beyond the distal end 134 of cannula 130 to reach surgical site 140.

Curved cannula 130 may include, for example, one or more straight portions 131 and one or more curved portions 133. In another example, curved cannula 130 may include a single curved portion (not shown). According to an exemplary embodiment, curved cannula 130 is a rigid cannula. Further, curved cannula 130 may be provided as a single piece, rigid cannula.

As shown in the exemplary embodiment of FIG. 1, a second patient side manipulator 150 may be provided, with a second instrument 152 and second curved cannula 154 mounted to second patient side manipulator 150. Second patient side manipulator 150, second instrument 152, and second curved cannula 154 may be arranged according to the exemplary embodiments described above. Second curved cannula 154, however, curves in a direction opposite to the direction in which curved cannula 130 curves, as shown in FIG. 1. Thus, two curved cannulas and associated instruments, curving in opposite directions, may be positioned to extend through a single incision 162 in the patient's bodywall 160 to reach surgical site 140. Further, although two curved cannulas and their respective instruments are shown in the exemplary embodiment of FIG. 1, a teleoperated surgical system may include one combination of curved cannula and instrument arranged according to the exemplary embodiments described above, three combinations of curved cannulas and instruments, or more combinations of curved cannulas and instruments.

According to an exemplary embodiment, each curved cannula may initially angle away from a straight line that extends between the incision 162 and the surgical site 140, and then curve back towards the line to direct the extended instruments to the surgical site 140. By operating patient side manipulators 120, 150 via pitch and yaw, the distal ends 134, 155 of the curved cannulas 130, 154 move accordingly, and therefore instrument end effectors 116, 156 are moved with reference to the surgical site 140 and, consequently, with reference to the endoscope's field of view. According to an exemplary embodiment, a remote center of motion (not shown) for second cannula 154 and second instrument 152 is proximate to the remote center of motion 142. Thus, the remote centers of motion for the two curved cannulas 120, 154 and their respective instruments 110, 152 are not identical, but are sufficiently close enough (proximate) to one another so that they can both be positioned at the single incision 162.

As shown in the exemplary embodiment of FIG. 1, a surgical image capture device, such as an endoscope 170 may be provided. Endoscope 170 may be mounted to a patient side manipulator 172 so that endoscope extends through the single incision 162, along with the two curved cannulas 130, 154 and their respective surgical instruments 110, 152. Endoscope 170 may extend through a conventional cannula 174 that is supported by cannula mount 176. Patient side manipulator 172 may be positioned to place a remote center of motion (not shown) of endoscope 170 at incision 162. As discussed above, it can be seen that the remote centers of motion for the two curved cannula 130, 154 and the endoscope 170 may not be identical, but may be positioned sufficiently close to allow the cannulas 130, 154, 174 to extend through the single incision 162 without the incision being made unduly large.

As shown in the exemplary embodiment of FIG. 1, the patient side manipulators 120, 150, 172 may be positioned so that each has a significantly improved volume in which to move in pitch and yaw without interfering with each other. That is, if straight-shaft instruments are used, then the patient side manipulators would need to be located in positions near one another to keep the shafts of instruments in a near parallel relation to effectively work through a single incision. However, using curved cannulas and instruments with flexible shafts permits the patient side manipulators to be placed farther apart so that each patient side manipulator may move within a relative larger volume than the volume provided when straight-shafted instruments are used. Further, the exemplary embodiment of FIG. 1 shows how the curved cannulas 130, 154 provide improved triangulation for the surgical instruments 110, 152, so that the surgical site 140 is relatively unobstructed in a field of view (encompassed by dashed lines 178) of endoscope 170.

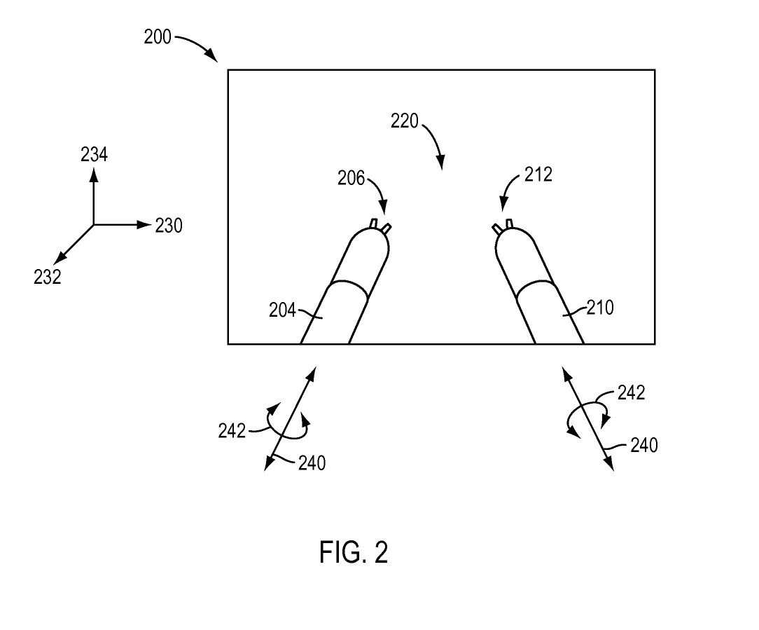

One consideration when utilizing a teleoperated surgical system with a configuration for a single opening is that surgical instruments may be oriented at a greater vertical angle relative to a surgical site than surgical instruments of a teleoperated surgical system configured for multiple openings. As a result, a view provided by an endoscope or other surgical image capture device may project along a length of surgical instruments and be oriented at a shallow angle to the surgical instruments. This is demonstrated in the exemplary embodiment of FIG. 2, which shows an exemplary view 200 provided by an endoscope or other surgical image capture device of a shaft 204 of a first surgical instrument, a shaft 210 of a second surgical instrument, and a surgical site 220. The endoscope, first surgical instrument, and second surgical instrument may be arranged according to endoscope 170 and surgical instruments 110, 152 of the exemplary embodiment of FIG. 1.

As shown in the exemplary embodiment of FIG. 2, a view 200 provided by an endoscope may project along a length of the shafts 204, 210. As a result, the instrument shafts 204, 210 may obstruct or partially obstruct a view of end effectors 206, 212. The degree to which instrument shafts 204, 210 may obstruct a view of end effectors 206, 212 may depend upon the geometry and type of instrument, with generally smaller end effectors being obstructed more than larger end effectors.

Wristed instruments may address this consideration by providing yaw and/or pitch movement to angle an end effector relative to a shaft of a surgical instrument and further into the view of a surgeon. However, as discussed above, the instruments of shafts 204, 210 may be non-wristed instruments. For instance, the instruments of shafts 204, 210 may lack a wrist that would otherwise provide a yaw or pitch movement of end effectors 206, 212 relative to the distal ends of the shafts 204, 210. The non-wristed instruments may include, for example, the following motions or degrees of freedom: translation of shafts 204, 210 in various directions along an X direction 230, Y direction 232, and Z direction 234 shown in the exemplary embodiment of FIG. 2; roll in a direction 242 relative to a longitudinal axis 240 of an instrument shaft; and actuation of end effectors 206, 212, such as opening and closing. Although the lack of a wrist advantageously simplifies the mechanical structure of a teleoperated surgical system, the lack of a wrist also can in some cases limit how end effectors 206, 212 may be oriented relative to instrument shafts 204, 210, such as to enhance the view of end effectors 206, 212.

To address this consideration, an end effector of a surgical instrument may be provided with a curved shape. A curved shape may be provided, for example, by an end effector having at least a portion with a curved longitudinal axis. A curved longitudinal axis may, for example, be a curved line that is not straight. A curved longitudinal axis may be provided, for example, by a portion of an end effector having a continuously curving longitudinal axis. In another example, two or more straight sections of an end effector oriented at an angle to one another may provide a curved longitudinal axis. However, a portion of an end effector having a curved longitudinal axis is not limited to these examples and may be provided by other configurations, such as by continuously curved sections oriented at angles to one another, a combination of continuously curved sections and straight sections oriented at an angle to one another, and other configurations.

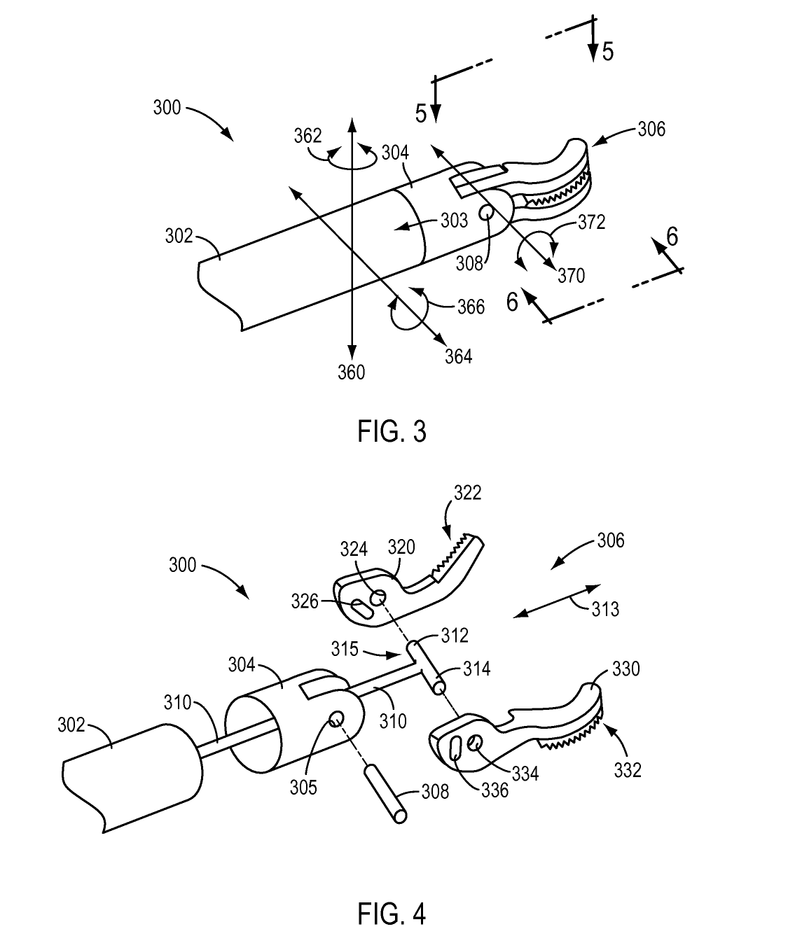

Turning to FIG. 3, an exemplary embodiment of a surgical instrument 300 for a teleoperated surgical system is shown that includes a shaft 302, clevis 304, and an end effector 306. As shown in FIG. 4, which is an exploded view of the exemplary embodiment of FIG. 3, end effector 306 may include a first jaw 320 and a second jaw 330, which are curved. First jaw 320 may include a grip portion 322, a connection aperture 324, and an actuation aperture 326. Similarly, second jaw 330 may include a grip portion 332, a connection aperture 334, and an actuation aperture 336.

According to an exemplary embodiment, connection apertures 324, 334 may be used to connect jaws 320, 330 to clevis 304, which is in turn connected to shaft 302. For instance, as shown in the exemplary embodiment of FIGS. 3 and 4, a pin 308 may be inserted through connection apertures 324, 334 and through an aperture 305 in clevis 304 to connect jaws 320, 330 to clevis. Pin 308 may also serve as an axis of rotation about which jaws 320, 330 rotate when end effector 306 is actuated to open and close jaws 320, 330, which will be described below.

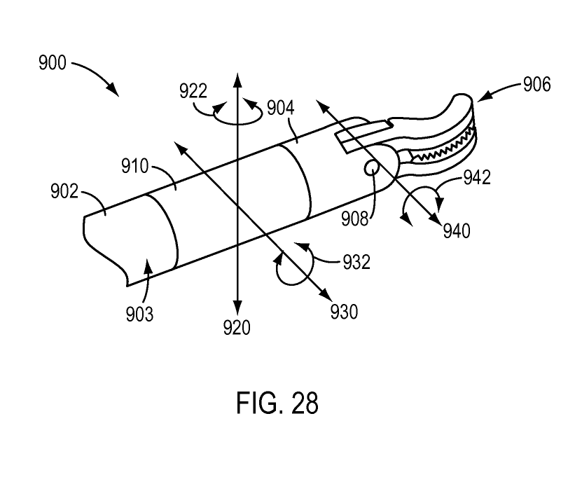

In various exemplary embodiments, a surgical instrument may include a wrist that provides at least one degree of freedom. A wrist may include, for example, two or more pieces linked together in a manner that permits relative movement between the pieces. For instance, with reference to FIG. 28, a surgical instrument 900 may include a shaft 902, clevis 904, and an end effector 906, as discussed above with regard to the exemplary embodiment of FIG. 3. Surgical instrument 900 may further include a jointed wrist 910 that provides movement with one or more degrees of freedom. According to an exemplary embodiment, wrist 910 may permit clevis 904 and end effector 906 to move relative to a distal end 903 of shaft 902, such as by moving in a pitch or yaw direction 922 (depending upon a which naming convention is selected for wrist movements) about axis 920. Another DOF could be provided by a jointed wrist to permit clevis 904 and end effector 906 to move relative to distal end 903 of shaft 902 by moving in a pitch or yaw direction 932 about axis 930, as shown in the exemplary embodiment of FIG. 28. Further, a surgical instrument 900 may be configured to have a DOF permitting end effector 906 to move relative to clevis 904, such as by rotating end effector 906 in direction 942 about an axis 940 passing through pin 908, which could provide similar motion as a wrist. A jointed wrist may provide any combination of one or more of these DOF's and other DOF's used in the art for jointed wrists in surgical instruments.

A surgical instrument may be a non-wristed instrument, such as surgical instrument 300 of the exemplary embodiment of FIG. 3. In contrast to an instrument with a wrist that provides one or more DOF's, a wrist portion of a non-wristed instrument may lack a linkage that permits relative movement between pieces of the instrument. A non-wristed instrument may, for example, provide zero DOF's at a wrist portion of the surgical instrument. For instance, a non-wristed surgical instrument 300 may be configured such that clevis 304 is connected to shaft 302 without a linkage to permit movement of clevis 304 and end effector 306 relative to distal end 303 of shaft 302. For example, a non-wristed instrument 300 may not be able to move end effector 306 and clevis 304 relative to distal end 303 of shaft 302 in a pitch or yaw direction 362 about axis 360 or in a pitch or yaw direction 366 about axis 364. In another instance, clevis 304 and end effector 306 may be connected without a linkage that would permit end effector 306 to move relative to clevis 304, such as in a pitch or yaw direction 372 about axis 370. According to an exemplary embodiment, a non-wristed surgical instrument 300 may have DOF's for translation of the instrument 300, rolling of shaft 302, and end effector actuation movement (such as opening and closing jaws), such as those described above for the exemplary embodiment of FIG. 2.

According to an exemplary embodiment, jaws 320, 330 may comprise a corrosion resistant alloy. For instance, jaws 320, 330 may comprise a stainless steel, such as, for example 17-4 PH stainless steel. In another example, jaws 320, 330 may comprise 17-4 PH stainless steel heat treated to have a hardness of 36-40 on the Rockwell C scale (36-40 HRC). According to an exemplary embodiment, grip portions 322, 332 of jaws 320, 330 may be provided as separate pieces joined to jaws 320, 330. For instance, grip portions 322, 332 may be provided as carbide inserts connected to the metal or alloy jaws 320, 330. The carbide inserts may comprise, for example, tungsten carbide. Carbide inserts may be joined to jaws 320, 330 via, for example, brazing or other joining methods using in the art to join carbide and metal. According to an exemplary embodiment, carbide inserts may be provided as a matched pair with complementary teeth that mesh together when the end effector is closed.

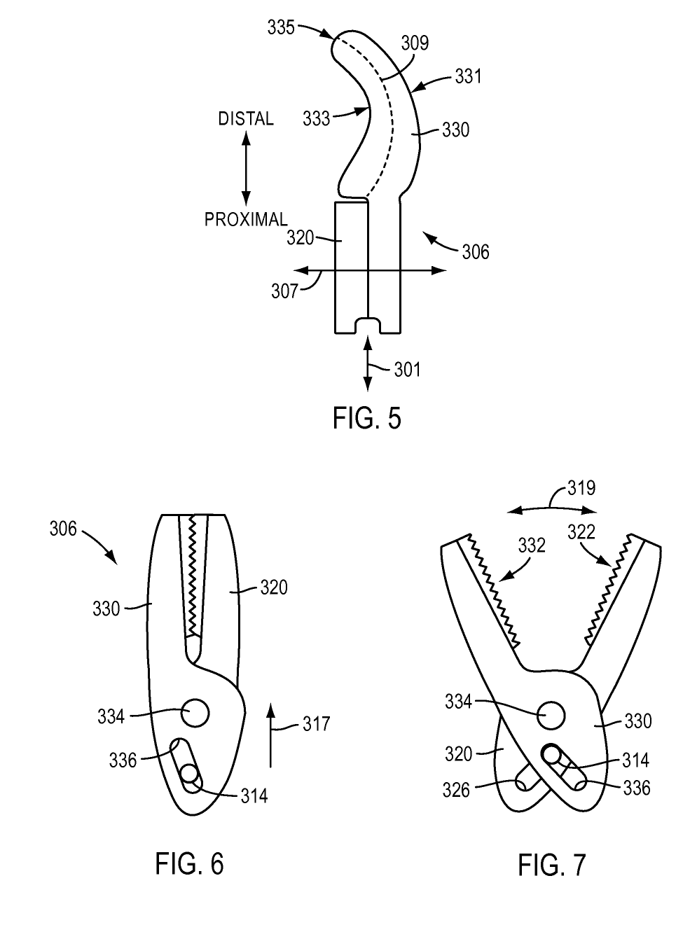

Turning to FIG. 5, a top view of end effector 306 is shown along line 5-5 in FIG. 3, without clevis 304, and with jaw 330 located on top of jaw 320. Although a majority of jaw 320 is not visible in FIG. 5, jaw 320 may have a complementary shape that substantially mirrors that of jaw 330. As shown in the exemplary embodiment of FIG. 5, jaw 330 may have a curved shape relative to a longitudinal axis 301 of a surgical instrument shaft 302. For instance, jaw 330 may curve relative to a longitudinal axis 301 of the surgical instrument shaft so that jaw 330 has an outer convex curved surface 331 and an inner concave curved surface 333. In another instance, at least a portion of end effector 306 has a curved longitudinal axis 309 between a proximal and distal end of the end effector 306, as shown in FIG. 5. Thus, only a portion of longitudinal axis 309 may be curved, such as along a portion of a length of jaws 320, 330 in a proximal-distal direction, or longitudinal axis 309 may be curved along its length, such as along an entire length of jaws 320, 330 in the proximal-distal direction. Further, the curvature of jaws 320, 330 may be oriented so that jaws 320, 330 curve in a plane that extends between the grip portions 322, 332, of jaws 320, 330, with the plane being parallel to the page of FIG. 5. According to an exemplary embodiment, an axis 307, which may represent a pivot axis of jaws 320, 330, extending through connection apertures 324, 334 (not shown in FIG. 5) and pin 308 (not shown in FIG. 5) may be substantially parallel to the plane of curvature.

Surgical instrument 300 may include a mechanism to actuate end effector 306, such as to open and close jaws 320, 330. Turning to FIG. 4, surgical instrument may include an actuation mechanism 310 connected to end effector 306. A proximal end (not shown) of actuation mechanism 310 may be connected to a patient side manipulator (not shown) of a teleoperated surgical system that provides motive force to actuation mechanism 310. For instance, actuation mechanism 310 may be a push/pull drive element rod that is pushed or pulled along direction 313 in FIG. 4 by a motive force provided by the patient side manipulator to actuate end effector 306. However, actuation mechanism 310 is not limited to a push/pull drive element and may include other types of drive elements, such as pull/pull drive elements and other actuators used in the art.

A distal end 315 of actuation mechanism 310 may be connected to end effector 306 to translate the motive force from the patient side manipulator to the jaws 320, 330. According to an exemplary embodiment, distal end 315 of actuation mechanism 310 may include a first projection 312 connected to jaw 320 and a second projection 314 connected to jaw 330. For instance, jaw 320 may include an actuation aperture 326 that first projection 312 is inserted into and jaw 330 may include an actuation aperture 336 that second projection 314 is inserted into. Actuation apertures 326, 336 may be in form of, for example, elongated slots, such as rectangular or oval slots, that projections 312, 314 may be inserted into. Thus, as actuation mechanism 310 is pushed or pulled along direction 313 in FIG. 4, projections 312, 314 may slide within actuation apertures 326, 336, causing jaws 320, 330 to pivot about pin 308.

Turning to FIG. 6, a side view of end effector 306 is shown along line 6-6 in FIG. 3 but without clevis 304 and pin 308. In the exemplary embodiment of FIG. 6, the jaws 320, 330 of end effector 306 are in a closed position. Although actuation mechanism 310 is not shown in FIG. 6, second projection 314 is shown within actuation slot 336 of jaw 330. When actuation mechanism 310 is pushed in direction 317 in FIG. 6, second projection 314 is forced upwards. Consequently, jaws 320, 330 rotate and pivot about pin (not shown) located in connection aperture 334 in direction 319 in FIG. 7, causing jaws 320, 330 to separate and move to an open position.

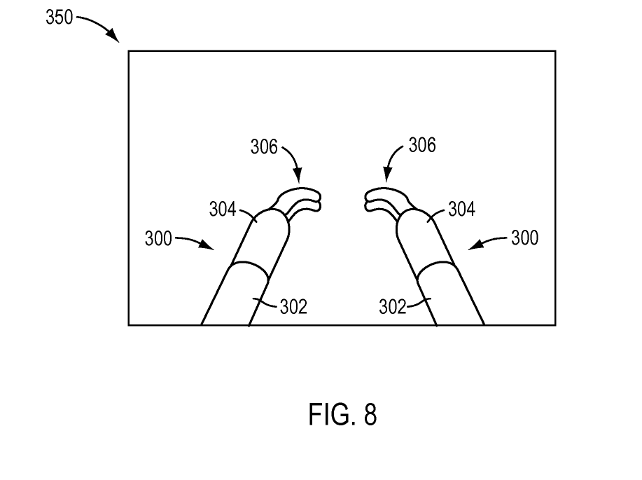

According to an exemplary embodiment, an end effector 306 of a surgical instrument 300 may have a shape selected to provide enhanced visibility of the end effector 306, particularly when surgical instrument 300 is non-wristed. For instance, end effector 306 may include jaws 320, 330 that are curved, as shown in FIGS. 3-5. Because jaws 320, 330 are curved, jaws 320, 330 may extend by a larger amount into a field of view during a surgical procedure. Turning to FIG. 8, a view 350 provided by an endoscope of a remote site is shown, with surgical instruments 300 having curved end effectors 306, as discussed in the embodiments of FIGS. 3-7, extending into view. As discussed above, surgical instruments 300 may each include a shaft 302, a clevis 304, and an end effector 306. Because the end effector 306 includes curved jaws, as shown in the exemplary embodiment of FIG. 8, end effector 306 may curve away from shaft 302 and clevis 304 of instruments 300 so that a view of end effectors 306 is enhanced as compared to an instrument that utilizes a straight end effector, such as in FIG. 2, for example.

The dimensions of jaws 320, 330 may be selected to provide jaws 320, 330 with a desired curvature. For example, outer curved surface 331 of jaw 330 in the exemplary embodiment of FIG. 5 may have a radius of curvature, for example, ranging from about 0.100 inches to about 0.300 inches. In another example, outer curved surface 331 may have a radius of curvature ranging, for example, from about 0.150 inches to about 0.250 inches. In another example, outer curved surface 331 may have a radius of curvature ranging, for example, from about 0.180 inches to about 0.220 inches. Inner curved surface 333 in the exemplary embodiment of FIG. 5 may have a radius of curvature ranging, for example, from about 0.100 inches to about 0.200 inches. In another example, inner curved surface 333 may have a radius of curvature ranging, for example, from about 0.130 inches to about 0.170 inches. According to an exemplary embodiment, tip 335 of jaw 330 may be rounded. For instance, tip 335 may have a radius of curvature ranging, for example, from about 0.050 inches to about 0.070 inches. As noted above, although a majority of jaw 320 is not visible in FIG. 5, jaw 320 may have a complementary shape that substantially mirrors that of jaw 330. Thus, jaw 320 may have an outer curved surface, inner curved surface, and rounded tip that matches those of jaw 330 in a complementary manner.

Shaft 302 of surgical instrument 300 may be flexible to permit instrument 300 to be advanced and withdrawn through a rigid curved cannula while being sufficiently stiff to provide effective surgical action at a surgical site when the end effector 306 and a distal end 303 of shaft 302 are projected beyond a distal end of the curved cannula, as discussed above in the exemplary embodiment of FIG. 1. However, because the cannula is curved and does not follow a straight line from end to end, friction between the cannula and instrument 300 may be considered when the instrument 300 is advanced or withdrawn through the cannula. In addition, the inner diameter and outer diameter of a curved cannula may vary from end to end of the cannula. In view of these considerations, it may be desirable to select dimensions of an end effector 306 that minimizes interference during advancing and withdrawing the end effector 306 through the curved cannula, while also providing a curved end effector 306 with enhanced visibility during use.

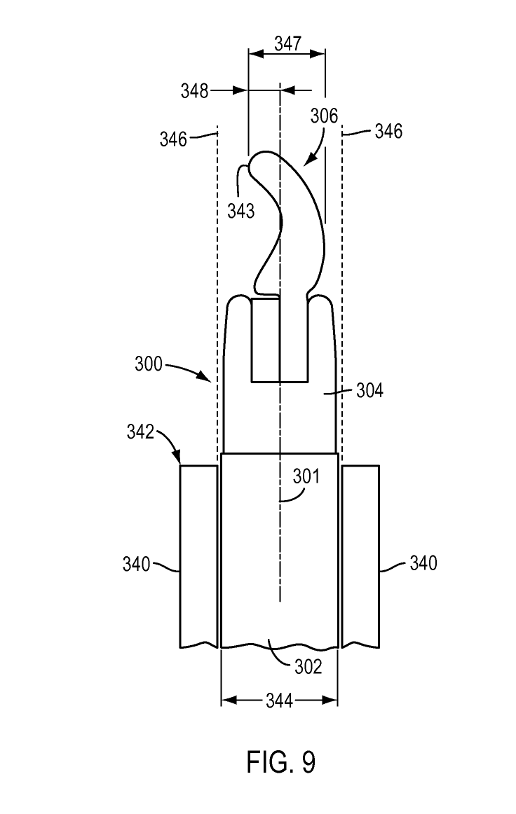

Turning to FIG. 9, surgical instrument 300 is shown within a rigid curved cannula 340, according to an exemplary embodiment. In FIG. 9, surgical instrument 300 has been advanced so that end effector 306, clevis 304, and a portion of shaft 302 extend beyond a distal end 342 of curved cannula 340. As shown in the exemplary embodiment of FIG. 9, an imaginary cylinder 346 may be projected from the distal end 342 of curved cannula 340. Cylinder 346 may have the same diameter as an inner diameter 344 of curved cannula 340 at the distal end 342 of cannula 340.

As shown in the exemplary embodiment of FIG. 9, the shape and curvature of end effector 306 may be selected so that end effector 306 remains within the imaginary cylinder 346 when instrument is extended beyond the distal end 342 of curved cannula 340, at least when end effector 306 is in a closed state. In other words, the total lateral dimension 347 of end effector 306 is equal to or less than the inner diameter 344 of curved cannula 340. By providing an end effector 306 that remains within imaginary cylinder 346, friction between end effector 306 and an interior surface of a curved cannula may be minimized when the end effector 306 is advanced and withdrawn through the cannula, which in turn may minimize wear of the end effector 306 and/or cannula.

As discussed above in the exemplary embodiment of FIG. 5, the dimensions and curvatures of jaws 320, 330 may be selected to provide end effector 306 with a desired shape. According to an exemplary embodiment, a throw distance 348, which may be a distance between a lateral tip 343 of end effector 306 and a longitudinal axis 301 of instrument 300, may be selected so that end effector 306 is provided with a predetermined shape that permits end effector 306 to remain within imaginary cylinder 346. According to an illustrative implementation, throw distance 348 may be affected by dimensions of end effector 306. For instance, when outer curved surface 331 of jaw 330 in the exemplary embodiment of FIG. 5 has a radius of curvature ranging from, for example, about 0.100 inches to about 0.300 inches and inner curved surface 333 of jaw 330 has a radius of curvature ranging, for example, from about 0.100 inches to about 0.200 inches, throw distance 348 may range, for example, from about 0.110 inches to about 0.050 inches. Further, because of the flexibility of shaft 302 and the predetermined shape of end effector 306, surgical instrument 300 may be inserted into a curved cannula without regard to the orientation of the end effector 306 to a curvature of the curved cannula. Thus, no steps or equipment is necessary to orient end effector 306 relative to the curvature of the curved cannula to minimize friction and/or ensure proper movement of the instrument through the cannula.

According to an exemplary embodiment, a predetermined shape of end effector 306 may be selected so that at least a portion of end effector 306 extends outside of the imaginary cylinder 348 when instrument 300 has been advanced to the position shown in FIG. 9. In this embodiment, end effector 306 may be oriented relative to the curvature of a curved cannula before instrument 300 is inserted into the cannula to permit end effector 306 to be advanced and withdrawn through cannula, and to extend outside of cylinder 348 when instrument 300 has been advanced past the distal end 342 of curved cannula 340.

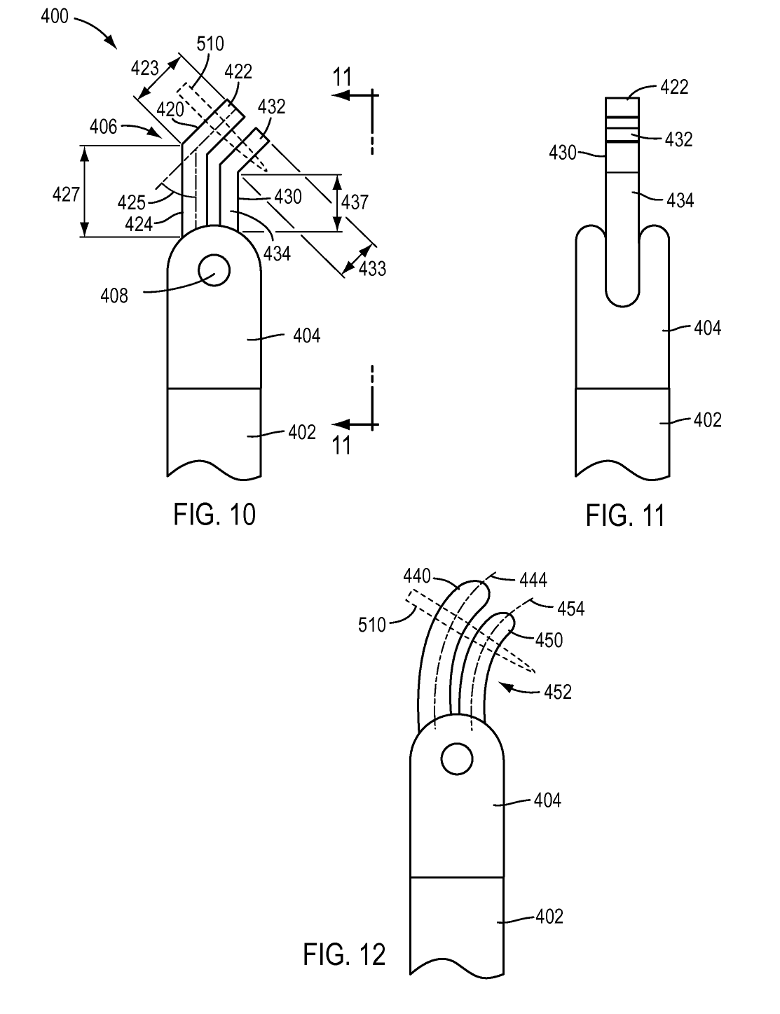

An end effector of a surgical instrument may have different shapes than those of the exemplary embodiments above. Turning to FIG. 10, an exemplary embodiment of a surgical instrument 400 is shown that includes a flexible shaft 402, a clevis 404, and an end effector 406. End effector 406 may include a first jaw 420 and a second jaw 430. FIG. 11 depicts a side view of jaws 420, 430, clevis 404, and shaft 402 along line 11-11 in FIG. 10. Jaws 420, 430 may be connected to clevis 404 via pin 408 and actuated as discussed in the exemplary embodiment of FIGS. 3-7 above. In contrast to the exemplary embodiment of FIGS. 3-7, the curvature of jaws 420, 430 may be oriented so that jaws 420, 430 curve in a plane that is parallel to the page of FIG. 10 but is substantially perpendicular to a plane that would extend between jaws 420, 430. Thus, an axis (not shown) extending through pin 408 and into the page of FIG. 10, which may represent a pivot axis for jaws 420, 430, would be substantially perpendicular to the plane of curvature for jaws 420, 430 in FIG. 10. As discussed above, a plane of curvature in the exemplary embodiment of FIG. 5 may be substantially parallel to a pivot axis 307 extending through pin 308. Thus, the plane of curvature in FIG. 10 may be substantially perpendicular to the plane of curvature in FIG. 5, according to an exemplary embodiment.

As shown in the exemplary embodiment of FIG. 10, the curvature of jaws 420, 430 may be provided by first sections 422, 432 that are oriented at an angle to second sections 424, 434 of jaws 420, 430. First sections 422, 432 and second sections 424, 434 may each be substantially straight and oriented at angle 425 relative to one another to provide jaws 420, 430 with a curvature, as shown in the exemplary embodiment of FIG. 10. Angle 425 may, for example, range from about 5° to about 85°. In another example, angle 425 may range from about 30° to about 60°. First sections 422, 432 may have lengths of 423, 433, respectively, and second sections 424, 434 may have lengths of 427, 437, respectively. Lengths 423, 433, 427, 437 may range, for example, from about 0.100 inches to about 0.150 inches. According to an exemplary embodiment, lengths 423, 433 of first sections 422, 432 may be longer than lengths 427, 437 of second sections 424, 434. According to another exemplary embodiment, lengths 427, 437 may be longer than lengths 423, 433. At least a portion of jaws 440, 450 may have curved longitudinal axes 444, 454. For instance, only a portion of jaws 440, 450 may have a curved longitudinal axis 444, 454 or the longitudinal axis 444, 454 may have a single curved section formed along the length of jaws 440, 450, as shown in FIG. 12, which may be a continuous curvature along the length of jaws 440, 450. For example, longitudinal axes 444, 454 may have a radius of curvature that ranges, for example, from about 0.100 inches to about 0.200 inches

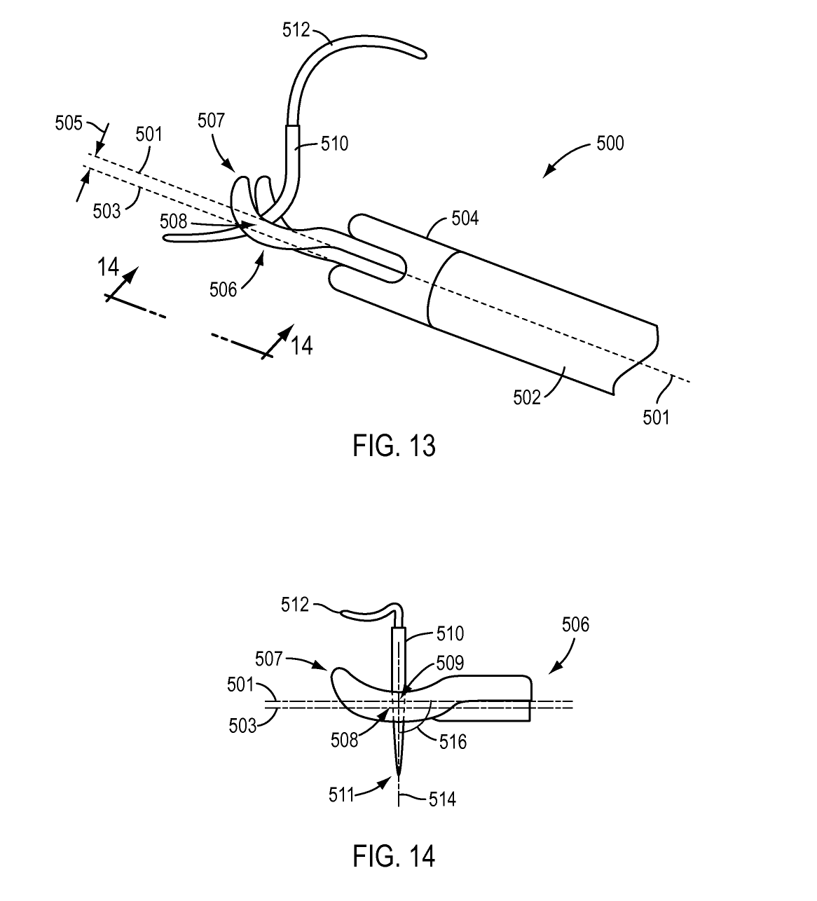

The shape of an end effector may affect how the end effector interacts with objects. For instance, the shape of an end effector may enhance the grasping force of an object. One type of surgical instrument that may be configured according to the embodiments described above is a needle driver. A needle driver may be used to grasp a needle during a suturing procedure. Turning to the exemplary embodiment of FIG. 13, a needle driver 500 is shown that can be used as a surgical instrument in a teleoperated surgical system. Needle driver 500 includes a flexible shaft 502, a clevis 504, and an end effector 506. As discussed above in regard to the exemplary embodiment of FIGS. 3-7, needle driver 500 may be a non-wristed instrument in various exemplary embodiments. Further, end effector 506 may be configured according to the exemplary embodiments of FIGS. 3-12.

End effector 506 of needle driver 500 may be configured to grasp a needle 510, as shown in the exemplary embodiment of FIG. 13. According to an exemplary embodiment, needle 510 may be a curved needle that a suture 512 is connected to. Needle 510 may be configured to be grasped by end effector 506. For instance, needle 510 may include one or more flat surfaces (not shown) to facilitate grasping of needle 510. According to an exemplary embodiment, needle 510 may be in the shape of a half circle along the length of the needle, with the half circle having a diameter of about 17 mm to about 36 mm. For instance, needle 510 may have an industrial size of, for example, RB-1 to CT-1, which may be manufactured by Ethicon, Inc. of Somerville, N.J. Suture 512 may have an industrial size of, for example, 0 to 4-0. Suture 512 may be a monofilament suture or have a multi-filament structure, according to an exemplary embodiment. Suture 512 may be a suture manufactured by Ethicon, Inc.

As shown in FIG. 13, end effector 506 may grasp needle 510 at a grip area 508. Due to the curvature of end effector 506, grip area 508 may be displaced relative to a longitudinal axis 501 of needle driver 500. For instance, with reference to FIGS. 13 and 14, an axis 503 passing through a center 509 of grip area 508 may be offset at a distance 505 from longitudinal axis 501. Distance 505 may range, for example, from about 0.110 inches to about 0.070 inches. When grip area 508 is offset at a distance 505 from longitudinal axis 501, a moment may be created when instrument 500 is rolled about longitudinal axis 501, which may advantageously enhance the force applied by end effector 506 to needle 510 during a suturing procedure.

FIG. 14 shows a side view of end effector 506, needle 510, and suture 512 along line 14-14 in FIG. 13 but without clevis 504 or shaft 502. As shown in FIG. 14, needle 510 may be grasped by end effector 506 so that an axis 514 extending along the length of needle 510 is oriented at an angle 516 that is substantially perpendicular to the longitudinal axis 501 of needle driver 500. However, grasping of a needle 510 by needle driver 500 is not limited to the configuration shown in FIGS. 13 and 14. For instance, a needle 510 may be grasped at different angles than those shown in FIGS. 13 and 14.

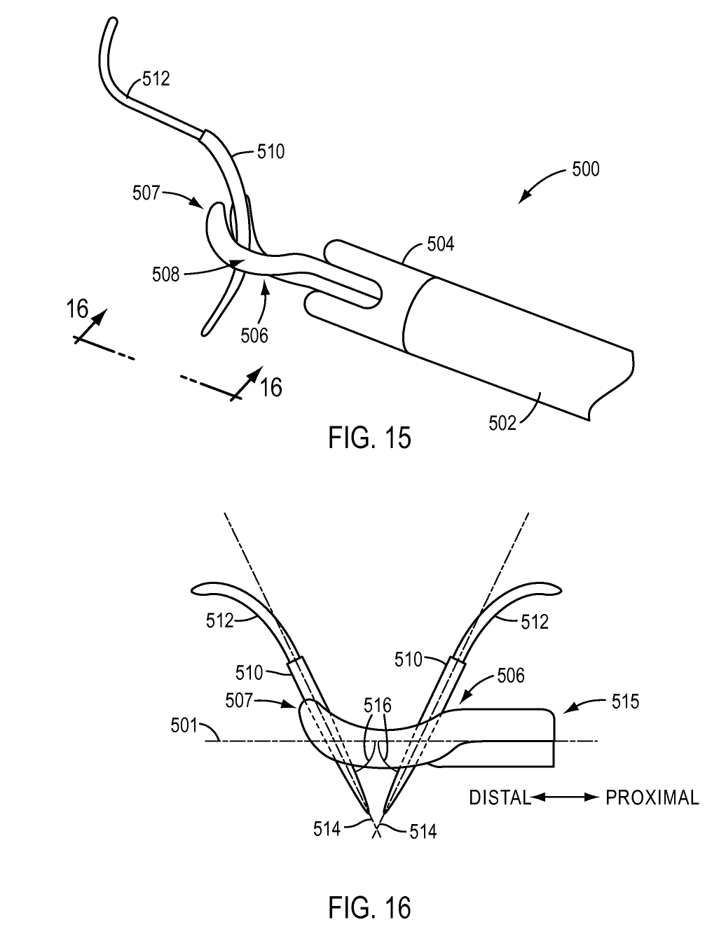

Turning to FIG. 15, needle 510 may be grasped by end effector 506 so that at least a portion of needle 510 is grasped along the curvature of end effector 506. As shown in FIG. 16, which shows a side view of end effector 506, needle 510, and suture 512 along line 16-16 of FIG. 15 but without clevis 504 or shaft 502, needle 510 may be grasped along a curvature of end effector 506 near distal tip 507 of end effector 506, as shown on the left hand side of FIG. 15, or needle 510 may be grasped along the curvature of end effector 506 near proximal end 515 of end effector 506. In either grasping configuration, needle 510 may be grasped by end effector 506 so that the axis 514 extending along the length of needle 510 is oriented at an angle 516 that is non-perpendicular to the longitudinal axis 501 of needle driver 500. In the configuration shown in the exemplary embodiment of FIGS. 15 and 16, angle 516 of either grasping configuration may range from about 30 degrees to about 70 degrees. Further, because at least a portion of needle 510 is grasped along the curvature of end effector 506, as shown in FIGS. 15 and 16, a grip area 508 may be increased in comparison to the grip area 508 shown in FIGS. 13 and 14. An increased grip area 508 can provide an increased amount of contact between end effector 506 and needle 510, which can thereby enhance the grip strength between end effector 506 and needle 510.

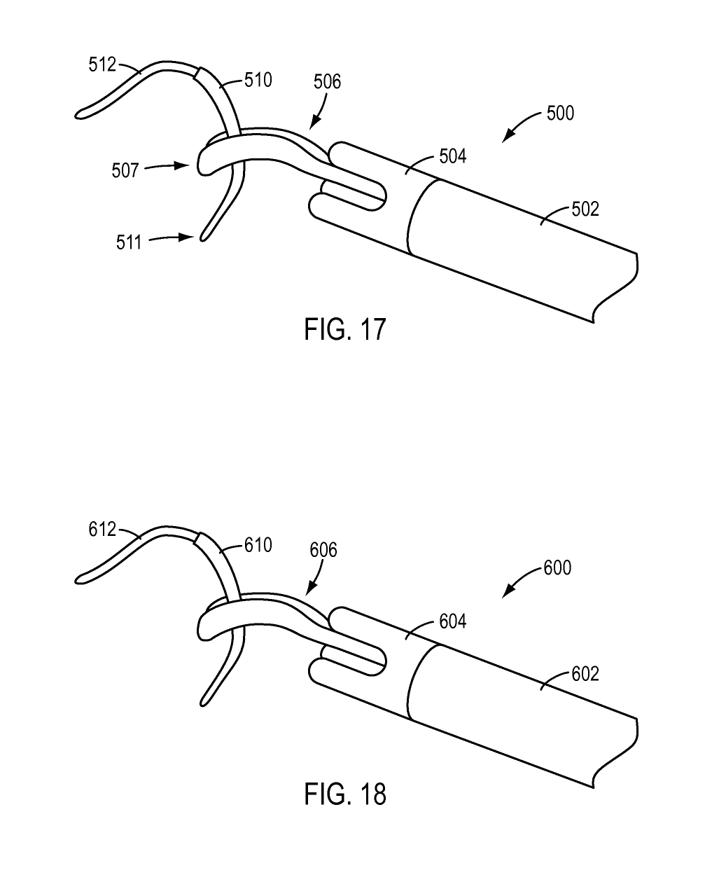

As shown in FIGS. 13-16, a needle 510 may be grasped by a needle driver 500 so that the convex curvature of end effector 506 is oriented substantially toward (e.g., faces generally in a direction of) a tip 511 of needle 510. Further, a tip 507 of end effector 506 may point or curve in a direction away from needle tip 511, as shown in FIGS. 13-16. However, grasping of needle 510 with end effector 506 is not limited to this configuration. Turning to FIG. 17, needle 510 may be grasped by end effector 506 so that the concave curvature of end effector 506 is oriented substantially toward (e.g., face generally in a direction of) needle tip 511. Further, tip 507 of end effector 506 may point or curve in a direction toward needle tip 511, as shown in FIG. 17.

In addition, although FIGS. 13-16 show the use of an end effector 506 having the configuration shown in the exemplary embodiment of FIGS. 3-7, any of the exemplary end effector embodiments discussed above may be used to grip a needle according to the exemplary embodiments discussed herein. For instance, a needle driver 600 including a shaft 602, clevis 604, and end effector 606 having the configuration of the exemplary embodiments of FIG. 10 may be used to grasp a needle 610 with a suture 612 according to the embodiments discussed above, as shown in the exemplary embodiment of FIG. 18. Further, a needle driver having an end effector (not shown) with a configuration of the exemplary embodiment of FIG. 12 may be used to grasp a needle according to the embodiments discussed above.

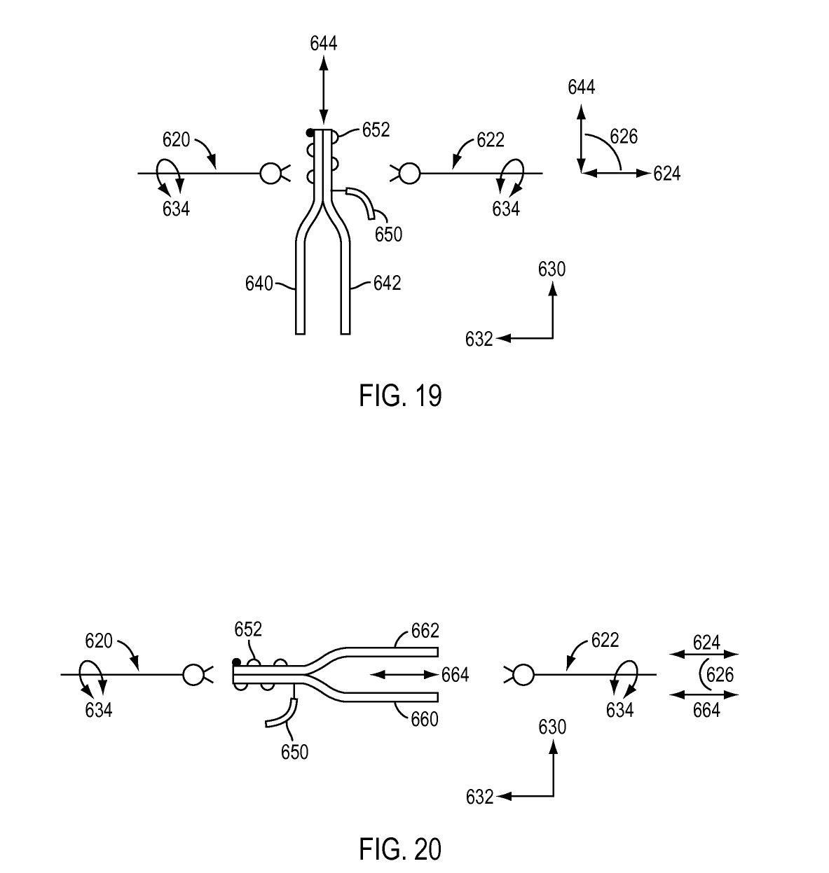

Turning to FIG. 19, a side view of a suturing procedure is shown in which a first surgical instrument 620 and a second surgical instrument 622 are being used to pass a needle 650 and suture 652 through a first tissue portion 640 and a second tissue portion 642 in a suturing operation. Instruments 620, 622 may be translated in an X direction 632 and/or in a Y direction 630 and may be rolled about a longitudinal axis 624 in direction 634. Further, if instruments 620, 622 include a wrist (not shown) with at least one DOF, the end effectors of instruments may be moved relative to the distal end of the shafts of the instruments, as discussed above in the exemplary embodiment of FIG. 3. Conversely, instruments 620, 622 may be non-wristed instruments, as discussed above in the exemplary embodiment of FIG. 3.

Because a long axis 644 of tissue portions 640, 642 is oriented at an angle 626 that is substantially perpendicular to the longitudinal axis of instruments 620, 622, suturing with instruments 620, 622 may be relatively easy, even if the movement of instruments 620, 622 is limited to directions 630, 632, 634. Further, angle 626 does not necessarily need to be approximately 90 degrees but instead may range, for example, from about 70 degrees to about 110 degrees.

Conversely, a first tissue portion 660 and a second tissue portion 662 may be oriented relative to the longitudinal axis 624 of instruments 620, 622 so that a long axis 664 of tissue portions 660, 662 forms an angle 626 to the longitudinal axis 624 that is relatively shallow, as shown in the exemplary embodiment of FIG. 20. For example, angle may range from about −20 degrees to about 20 degrees. When instruments 620, 622 are oriented relative to tissue portions 660, 662 at a relatively shallow angle, as shown in FIG. 20, suturing is more difficult than for an orientation shown in the exemplary embodiment of FIG. 19, particularly when movements of instruments are limited to directions 630, 632, 634. As discussed above with regard to the exemplary embodiment of FIG. 2, visibility of an end effector, as well as an object grasped by the end effector, may be hindered by the shaft of an instrument.

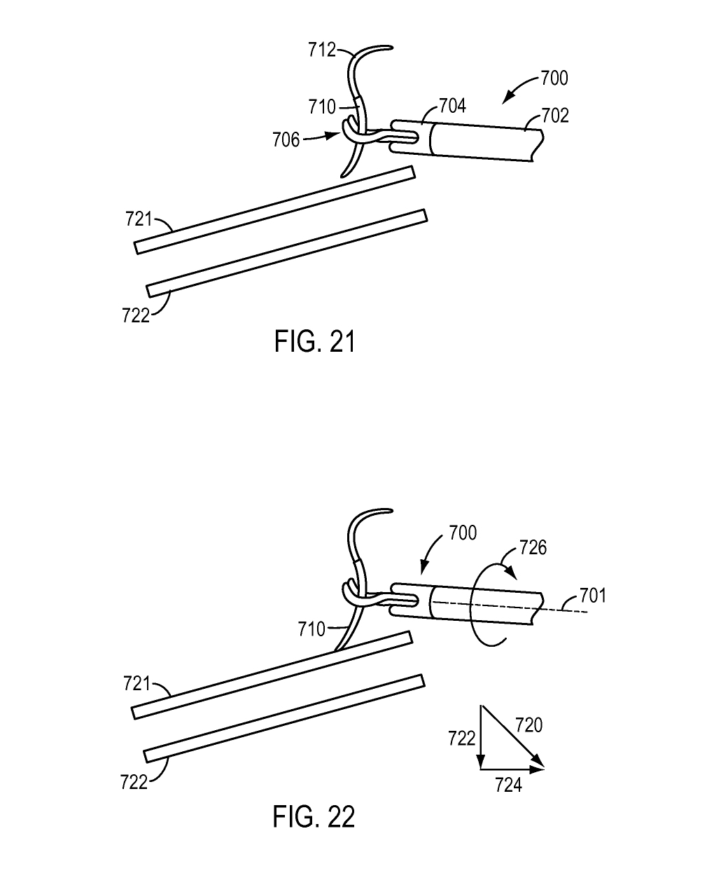

Surgical instruments using the curved end effector configurations discussed above may facilitate suturing procedures. As discussed above, a curved end effector may enhance visibility of an end effector and objects grasped by the end effector, as well as enhancing contact area between grips of an end effector and an object grasped. Various techniques may be used to move a needle that is grasped by a needle driver during a suturing procedure. An exemplary embodiment of a method of suturing is shown in FIGS. 21-24. Turning to FIG. 21, a needle driver 700 may include a shaft 702, a clevis 704, and an end effector 706 that is grasping a needle 710 with a suture 712. End effector 706 may be configured according to the exemplary embodiments of FIGS. 3-7 and 10-12 described above and may grasp needle 710 according to the exemplary embodiments of FIGS. 13-18 described above.

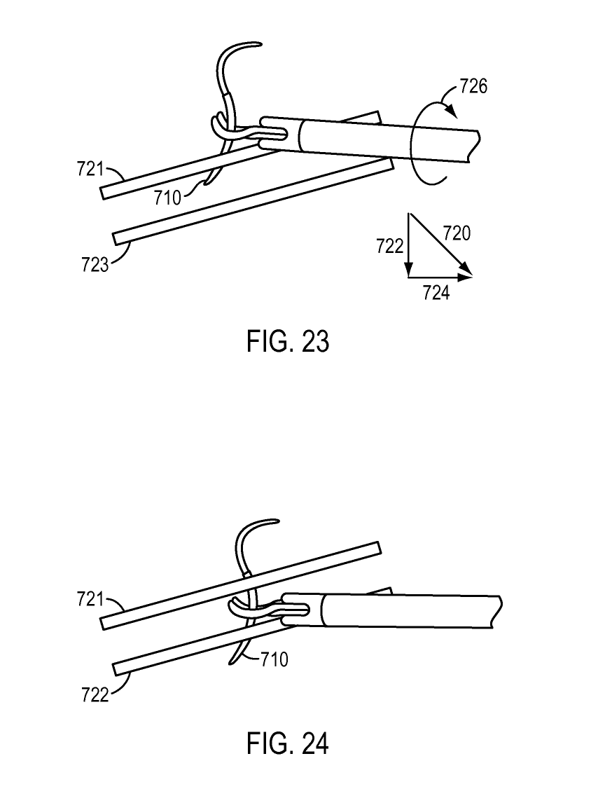

When suturing a first tissue portion 721 and a second tissue portion 723 together, needle driver 700 may be moved in a motion to drive needle 710 through the tissue portions 721, 723. As shown in the exemplary embodiment of FIG. 22, needle driver 700 may be translated in a direction 720 to initially drive needle 710 through first tissue portion, as shown in FIG. 23. Movement of needle driver 700 in direction 720 may be provided, for example, by a combination of a vertical movement along direction 722 and a horizontal movement along direction 724, with the vertical and horizontal notations being relative to the page of FIGS. 22 and 23. In addition, needle driver 700 may be rolled about the longitudinal axis 701 of needle driver 700, such as in direction 726 in FIGS. 22 and 23. For instance, when needle driver 700 is a non-wristed instrument, rolling may facilitate driving needle 710 and pulling suture 712 through tissue portions 721, 723 in a limited amount of space. As shown in FIGS. 23 and 24, needle driver 700 may continue to be driven along direction 720 (and optionally rolled in direction 726) to drive needle through second tissue portion 723.

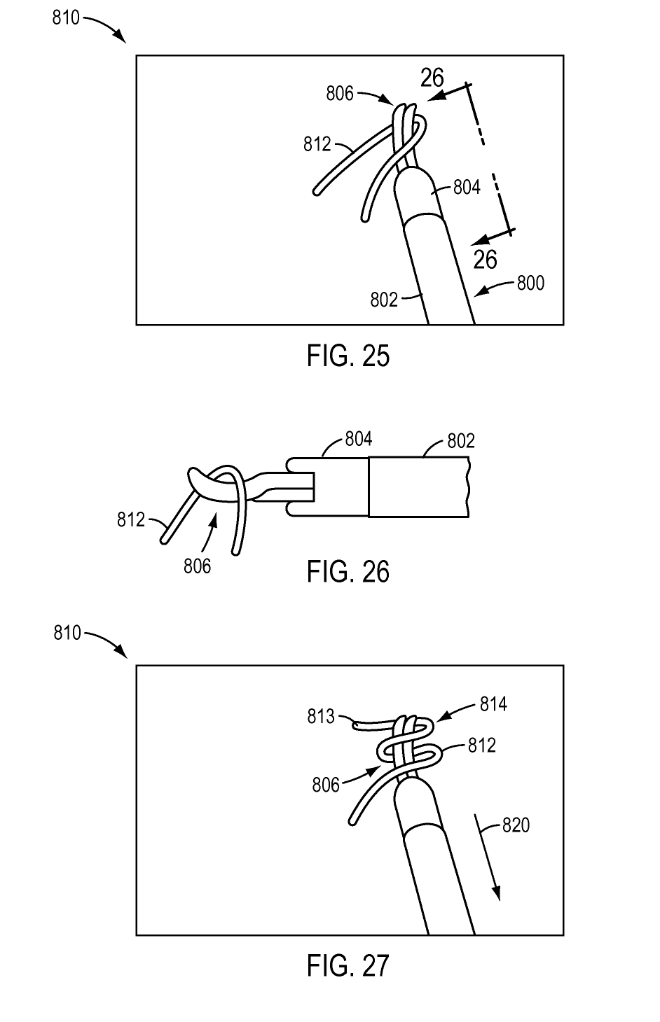

The predetermined shape of a needle driver end effector may facilitate performing a suturing procedure. Turning to FIG. 25, a view 810 of a needle driver 800 is shown during a knot-tying portion of a suturing procedure. Needle driver 800 includes a shaft 802, a clevis 804, and an end effector 806, which may be configured according to the exemplary embodiments of FIGS. 3-7 and 10-12 discussed above. As shown in FIG. 25, a suture 812 may be looped around needle driver 800 during the knot-tying operation. When the suture 812 is looped around needle driver 800, the curvature of end effector 806 may assist with maintaining the position of the suture 812 on the needle driver 800. As shown in FIG. 26, which is a side view of needle driver 800 and suture 812 along line 26-26 of FIG. 25, suture 812 may advantageously rest within the curve of end effector 806. In comparison to a needle driver that has a straight end effector, curved end effector 806 may reduce the occurrence of suture 812 sliding off end effector 806, which would require restarting the process of forming a loop to make a knot in suture 812. As shown in FIG. 27, once suture 812 is looped around needle driver 800, end effector 806 may be used to grasp a distal end 813 of suture and then pull distal end 813 along direction 820 and through loop 814 of suture 812 to form a knot.

By providing an end effector of a surgical instrument for a teleoperated surgical system with a curved shape (e.g. at least a portion having a curved longitudinal axis or otherwise non-straight longitudinal axis), visibility of the end effector may be enhanced during a surgical procedure. In addition, the curved shape of the end effector may enhance the grasping of objects, such as a needle or suture.

Further modifications and alternative embodiments will be apparent to those of ordinary skill in the art in view of the disclosure herein. For example, the systems and the methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

In this description, an actively flexible piece may be bent by using forces inherently associated with the piece itself. For example, one or more tendons may be routed lengthwise along the piece and offset from the piece's longitudinal axis, so that tension on the one or more tendons causes the piece to bend. Other ways of actively bending an actively flexible piece include, without limitation, the use of pneumatic or hydraulic power, gears, electroactive polymer, and the like. A passively flexible piece is bent by using a force external to the piece. An example of a passively flexible piece with inherent stiffness is a plastic rod or a resilient rubber tube. An actively flexible piece, when not actuated by its inherently associated forces, may be passively flexible. A single component may be made of one or more actively and passively flexible portions in series.

This description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

It is to be understood that the particular examples and embodiments set forth herein are nonlimiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their full breadth including equivalents.

What is claimed is:

1. A teleoperated surgical system, comprising:
a rigid cannula, wherein at least a portion of the cannula has a curved longitudinal axis; and
a surgical instrument, comprising:
a force transmission mechanism configured to engage a patient side manipulator of the surgical system;
a flexible shaft; and
an end effector coupled to the shaft, wherein the end effector comprises a pair of jaws, and at least a portion of each jaw has a curved longitudinal axis.

2. The teleoperated surgical system of claim 1, wherein the surgical instrument is a non-wristed instrument.

3. The teleoperated surgical system of claim 1, wherein the instrument further comprises a wrist having at least one degree of freedom.

4. The teleoperated surgical system of claim 1, wherein the pair of jaws open and close about a pivot axis, and wherein the curved longitudinal axis of each jaw portion curves in a plane that is substantially parallel to the pivot axis.

5. The teleoperated surgical system of claim 1, wherein the pair of jaws open and close about a pivot axis, and wherein the curved longitudinal axis of each jaw portion curves in a plane that is substantially perpendicular to the pivot axis.

6. The teleoperated surgical system of claim 5, wherein at least one of the jaws includes a first straight section and a second straight section, wherein the first straight section and the second straight section are oriented at an angle relative to one another.

7. The teleoperated surgical system of claim 1, wherein the flexible shaft is passively flexible.

8. The teleoperated surgical system of claim 1, wherein, in a position in which the end effector is located beyond a distal end of the cannula, the end effector remains within an imaginary cylinder, wherein the imaginary cylinder extends from and has a diameter substantially the same as an inner diameter of the cannula at the distal end of the cannula.

9. A surgical instrument, comprising:
a force transmission mechanism configured to engage a patient side manipulator of a teleoperated surgical system;
a flexible shaft; and
an end effector coupled to the shaft, wherein the end effector comprises a pair of jaws, and wherein at least a portion of each jaw has a curved longitudinal axis.

10. The surgical instrument of claim 9, wherein the surgical instrument is a non-wristed instrument.

11. The surgical instrument of claim 9, wherein the pair of jaws open and close about a pivot axis, and wherein the curved longitudinal axis of each jaw portion curves in a plane that is substantially parallel to the pivot axis.

12. The surgical instrument of claim 9, wherein the pair of jaws open and close about a pivot axis, and wherein the curved longitudinal axis of each jaw portion curves in a plane that is substantially perpendicular to the pivot axis.

13. The surgical instrument of claim 9, wherein the jaws of the end effector are configured to provide a grip area for grasping a needle, wherein an axis passing through a center of the grip area is offset from a longitudinal axis of the instrument.

14. A teleoperated surgical system, comprising:
a surgical instrument, wherein the instrument comprises:
a force transmission mechanism configured to engage a patient side manipulator of the teleoperated surgical system;
a flexible shaft; and
an end effector coupled to the flexible shaft, wherein the end effector includes a pair of jaws, wherein at least a portion of each jaw has a curved longitudinal axis; and
a needle configured to be grasped by the jaws.

15. The teleoperated surgical system of claim 14, wherein the jaws are configured to grasp the needle so that an axis extending along a length of the needle is oriented at an angle ranging from about 30 degrees to about 70 degrees relative to a longitudinal axis of the instrument.

16. The teleoperated surgical system of claim 14, wherein the jaws of the end effector are configured to provide a grip area for grasping the needle, wherein an axis passing through a center of the grip area is offset from a longitudinal axis of the instrument.

17. The teleoperated surgical system of claim 14, wherein the jaws of the end effector are configured to provide a grip area for grasping the needle, wherein the grip area is configured such that a curvature of the jaws orients a tip of the end effector to point away from a tip of the needle.

18. The teleoperated surgical system of claim 15, wherein the jaws of the end effector are configured to provide a grip area for grasping the needle, wherein the grip area is configured such that a curvature of the jaws orients a tip of the end effector to point toward a tip of the needle.
